(19) United States
(12) Patent Application Publication
(10) Pub. No.: US 2019/0357771 A1
(43) Pub. Date: Nov. 28, 2019

(54) SYSTEMS AND METHODS FOR DELIVERING, ELICITING, AND MODIFYING TACTILE SENSATIONS USING ELECTROMAGNETIC RADIATION

(71) Applicant: Pine Development Corporation, Mountain View, CA (US)

(72) Inventors: William J. Yu, Mountain View, CA (US); Alexander A. Brownell, Bountiful, UT (US)

(21) Appl. No.: 16/405,985

(22) Filed: May 7, 2019

Related U.S. Application Data
(60) Provisional application No. 62/668,155, filed on May 7, 2018.

(51) Int. Cl. A61B 5/00; A61N 5/06

(52) U.S. Cl. CPC A61B 5/0048; A61N 2005/063; A61N 5/0613

(57) ABSTRACT

The present disclosure pertains to systems and methods for directly and/or indirectly eliciting sensations utilizing electromagnetic radiation. In some embodiments, systems and methods for stimulation of excitable tissues using wavelengths of electromagnetic spectrum for inducing perceived cutaneous sensations are described. The systems and methods described enhance stimulation of the tissue. Utilizing these system and methods allows for increased control.

### RELATED APPLICATIONS

This application claims the benefit under 35 U.S.C. § 119(e) to U.S. Provisional Patent Application No. 62/668,155, filed May 7, 2018 and titled “Systems and Methods for Delivering, Eliciting, and Modifying Tactile Sensations Using Electromagnetic Radiation,” which is hereby incorporated by reference herein in its entirety.

### TECHNICAL FIELD

The present disclosure is directed to systems and methods for directly or indirectly eliciting sensations using electromagnetic radiation. More particularly, but not exclusively, the present disclosure is related to systems and methods for stimulation of excitable tissues using wavelengths of the electromagnetic spectrum for inducing perceived cutaneous sensations.

### BRIEF DESCRIPTION OF THE DRAWINGS

Non-limiting and non-exhaustive embodiments of the disclosure are described herein, including various embodiments of the disclosure illustrated in the figures listed below.

FIG. 1A illustrates a block diagram of a stimulation system using light emitting diodes (LEDs) as a source for electromagnetic radiation directed onto a user's tissue to elicit a tactile sensory response consistent with embodiments of the present disclosure.

FIG. 1B illustrates alternative arrangements of LEDs that may be utilized in order to achieve different capabilities in stimulating the tissue consistent with embodiments of the present disclosure.

FIG. 2A illustrates a conceptual representation of a rotary encoder that may be connected to a stimulation control system and used for titration of the sensation to a user's preference consistent with embodiments of the present disclosure.

FIG. 2B illustrates an example conceptual representation of an end-user feedback device in the form of a rocker switch and a button consistent with embodiments of the present disclosure.

FIG. 3A illustrates a conceptual representation of a finger sleeve device attached to a user's fingernail consistent with embodiments of the present disclosure.

FIG. 3B illustrates a cutaway view of the finger sleeve device and illustrates that the sleeve that is in contact with the dorsal side of the user's finger and separated from the palmar side of the finger consistent with embodiments of the present disclosure.

FIG. 3C illustrates an example attachment scheme of the finger sleeve device, in which there may or may not be adhesive over the nail, and there is an additional point of attachment where the intermediate phalanx is encircled by the open end of the sleeve such that the sleeve holds in place on the finger consistent with embodiments of the present disclosure.

FIG. 4A illustrates an example conceptual representation of a glove comprising a light-based tactile stimulation system consistent with embodiments of the present disclosure.

FIG. 4B illustrates an example conceptual representation of a glove comprising a light-based tactile stimulation system in which there is a contact interface for stimulation of the index finger consistent with embodiments of the present disclosure.

FIG. 4C illustrates an example conceptual representation of a glove comprising a light-based tactile stimulation system in which a stimulation module is separated from the skin by inflating and inserting internal supports consistent with embodiments of the present disclosure.

FIG. 4D illustrates an example conceptual representation of a glove comprising a light-based tactile stimulation system in which a plurality of stimulation modules are placed in several locations on a single finger consistent with embodiments of the present disclosure.

FIG. 5A illustrates a conceptual representation of a computer mouse that includes a tactile stimulation system consistent with embodiments of the present disclosure.

FIG. 5B illustrates a side-view cutaway view of the computer mouse and some of the internal components associated with a tactile system consistent with embodiments of the present disclosure.

FIG. 5C illustrates a top view of the computer mouse where an aperture for optical stimulation and a proximity sensor are visible, consistent with embodiments of the present disclosure.

FIG. 5D illustrates a top-view cutaway of the internal components of the computer mouse consistent with embodiments of the present disclosure.

FIG. 6A illustrates a conceptual representation of a virtual hand in contact with a virtual cube, in which fingers L2 (left index), L3 (left middle) and L4 (left ring) are all in contact with the virtual cube and would each receive sufficient stimulation to elicit sensation felt across all 3 of these fingers consistent with embodiments of the present disclosure.

FIG. 6B illustrates the virtual hand and virtual cube, in which only L3 remains in virtual contact with the cube and would therefore be the only finger to receive stimulation consistent with embodiments of the present disclosure.

FIG. 6C illustrates a virtual hand in contact with the virtual cube, in which the fingers are in close proximity and the hand is moved to laterally relative to the cube so that L2 remains in virtual contact with the cube and is to be stimulated consistent with embodiments of the present disclosure.

FIG. 7A illustrates an embodiment where a user is wearing a VR headset, which is coordinated with a tactile stimulation system consistent with embodiments of the present disclosure.

FIG. 7B illustrates an embodiment where a tactile stimulation system is attached directly to the VR headset and worn by the user allowing stimulation directed outward from the user's body onto his peripheral tissue consistent with embodiments of the present disclosure.

FIG. 7C illustrates the tactile stimulation system, that may be mounted to AR goggles, VR goggles, MR goggles, or other non-vision occluding projection systems consistent with embodiments of the present disclosure.

FIG. 8A illustrates a side-view diagram of a non-contact, free-space tactile stimulation system consistent with embodiments of the present disclosure.

FIG. 8B illustrates a front view of the optics of the non-contact, free-space tactile stimulation system consistent with embodiments of the present disclosure.

In the following description, numerous specific details are provided for a thorough understanding of the various embodiments disclosed herein. The systems and methods disclosed herein can be practiced without one or more of the specific details, or with other methods, components, materials, etc. In addition, in some cases, well-known structures, materials, or operations may not be shown or described in detail in order to avoid obscuring aspects of the disclosure. Furthermore, the described features, structures, or characteristics may be combined in any suitable manner in one or more alternative embodiments.

### DETAILED DESCRIPTION

According to various embodiments, perceived sensations may be produced by directing one or more wavelengths of electromagnetic radiation in both the visible and infrared spectrum, hereafter referred to as light, at the skin in a controlled manner. This may be done for the purposes of informing the user of such a system of some information, simulating the touch of an object, or creating novel sensations. The sensations may or may not mimic reality. Various embodiments consistent with the present disclosure relate to systems and methods that stimulate a user's tissues, either with or without direct contact with a physical interface by selectively directing electromagnetic radiation onto the tissue. Various embodiments of the present disclosure may be applied in a variety of fields, including virtual reality (VR), augmented reality (AR), mixed reality (MR), and holography. When objects are virtual, the interaction with such is commonly mediated through some device such as a keyboard, mouse, handheld controller, hand tracking system, etc. These interactions may lack a tactile experience consistent with or desirable for the interaction with a virtual object. When a user can insert a representation of his or her own body into the virtual, augmented, or holographic world, it may prove beneficial to interact with virtual objects in a manner similar to the physical world. Touching is a natural interrogation of objects, and feeling imparts knowledge about the object's characteristics. Touch can also elicit emotions in the user. In a virtual world, it could be valuable to induce sensations in the user to convey information about the object as well as to confirm contact. AR and MR may also benefit from providing confirmation of contact with a virtual object, by inducing sensations for interaction with a virtual object, or supplementing sensation on top of that provide by a real object. Holographic representations or holograms may also benefit from tactile sensations being delivered when the user directly interacts with a 3D or volumetric image with their hand or other body parts.

Tactile sensations can also be desirable for 2-D display technology such as that delivered through a visual display. Objects that are displayed can be virtual or non-virtual objects. In the case of a non-virtual object, such as a photo of an actual dress or a piece of fabric, the user may benefit from tactile sensations that are either intended to mimic the actual surface or other object properties of the real object, or an author's own generated tactile content. In one specific application, such sensations may provide improved experience to individuals shopping for clothing or other merchandise through the Internet. In another specific application, such sensations may provide an improved ability to utilize computer-aided drafting (“CAD”) software by enabling a designer to interact with a design in a new and novel ways.

It is the way in which the light is applied and controlled to the tissue that controls the quality and intensity of the sensation. In various embodiments, pulses of light in various widths, intensities, frequencies, angles of incidence, and spot sizes, among others, may be used to control the sensation. Various embodiments consistent with the present disclosure may tracking previously stimulated areas for the purpose of modulating a future stimulus. Systems and methods consistent with the present disclosure may deliver stimuli so that a consistent, intentional sensation may be felt in a plurality of ways with variations in actuator response or user tissue position.

The introduction of sensation to the user in conjunction with a VR, AR, MR, or holographic system consistent with the present disclosure may coordinate a visual or audio system and a tactile stimulation system. Coordination may comprise ensuring that the relative timing of the systems creates a consistent sensory experience. There may be some visual indication of a virtual contact with the object in addition to the tactile stimulation. In addition, there may be an auditory indication and/or a vibration or physical force delivered in conjunction with the tactile stimulation to augment the tactile sensation. Some information about the virtual, augmented, mixed, or holographic world may be delivered to the tactile stimulation system to limit the volume in space where tactile stimulation will occur.

In various embodiments, novel interactions with virtual and/or holographic objects may be created. Such interactions may provide additional information to users about the virtual and/or holographic object. Various embodiments consistent with the present disclosure may incorporate a physics model in an VR, AR, MR and/or holographic system and may coordinated tactile stimulation to match closely, or completely differ from, the physical world.

In some embodiments, a system is described herein consisting of one or more light sources that, when directed onto the skin of a user, may elicit various types of sensation as desired to communicate a particular tactile effect. The direction of multiple light sources may be accomplished by directly moving a single light source, reflecting the light, or using a plurality of light sources. Any number of actuators may be used to accomplish the various movements of the light. Described herein is the result of the direction of the light.

First is described a system of two distinct wavelengths of incident light directed onto the tissue. Three or more distinct wavelengths may also be used. The application of multiple wavelengths may accomplish one or more distinct goals such as the induction of different sensations simultaneously, modulation of a single type of sensation, intensification of a single type of sensation, maintaining a sensation, visible indication of stimulation site, or distraction away from a stimulation site. Stimulation protocols for the wavelengths may be identical, or more often, unique to each. The stimulation protocols modulate any or all of the following parameters, such as intensity or fluence, output power, pulse width, pulse profile, frequency, duty cycle, stimulation duration, time between stimuli, and spot size, to achieve the desired sensation. These modulations may be done separately or together to each of the applied wavelengths to achieve a variety of sensations. As such, the temporal coincidence of different wavelengths may change relative to one another. The multiple wavelengths may be spatially coincident or offset from one another.

The skin of a user provides the topological target of the various beams of light for stimulation. Spatial colocation of the various wavelengths proves useful for creating certain effects, while directing the stimuli to separate locations induces other types of sensations. Each of the beams may be steered independently or relative to one another by one or more actuators or beam steering mechanisms, including, but not limited to, microelectromechanical systems (MEMS) mirrors, digital micromirror device (DMD) used in digital light processing (DLP) projectors, and spatial light modulators (SLMs). The beams may also be modulated such that the area of stimulation on the skin changes in shape and or size either simultaneously or independently such as, but not limited to, a focusing lens system.

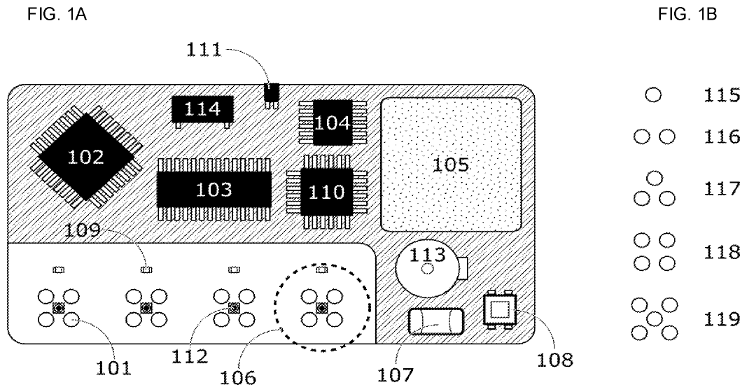

FIG. 1A illustrates a block diagram of a stimulation system using light emitting diodes (LEDs) as a source for electromagnetic radiation directed onto a user's tissue to elicit a tactile sensory response consistent with embodiments of the present disclosure. In this embodiment, system may be comprised of a series of LEDs 101 arranged to stimulate certain fingers, a control system 102, an LED driver 103, a power management IC 104, and a power source 105.

The system controller may be any controller technology including a microprocessing unit, graphics processing unit (GPU), memory chip, application specific integrated circuit chip (ASIC), a wireless communication chip with processing capabilities, a computer, a gaming console, a VR, AR, or MR device, a holographic imager, a mobile phone, or any other processor capable of communicating with the other components of the system to control the emission of desired stimulation. The LED driver 103 may alternatively be replaced or used in combination with any appropriate components, including multiplexers and de-multiplexers, potentiometers, and voltage and current regulators. Wireless communication chips may include Bluetooth, Zigbee, Z-Wave, Wi-Fi, or any other suitable technology. In some embodiments, the control system may work autonomously with user input. In some embodiments, the control system may work in coordination with other systems. In some embodiments, the control system may work autonomously with user input and in coordination with other systems.

In some embodiments, power source 105 may include a battery to be used when the device is used without any cords that might tether its use to a certain location or device. In some embodiments, power source 105 may use another suitable source of power. In another embodiment, the power source 105 may be the device in which the LED stimulation system may be incorporated or embedded. This device may be a mouse, game controller, or hand-tracking device. In some embodiments, power source 105 may be delivered to the LED stimulation system through a wired connection, for example, a USB cable.

The LEDs 101 are arranged in a square pattern with a certain spacing 106. The LEDs 101 may be arranged in a different pattern. The four LEDs 101 pictured here are grouped such that all four LEDs 101 are intended to illuminate a single tissue site such as, but not limited to, a finger, with the other arrangements targeting different tissue sites such as different fingers. Such groupings may have any or all of the individual LEDs 101 illuminated together or separately to achieve the desired tactile sensation. The arrangement of the LEDs 101 in this system is shown to be static. In some embodiments, the placement of the LEDs 101 may be changed by the end user. In this shown embodiment, LEDs 101 are described. In some embodiments, a different suitable light source may be utilized in place of the LED, such as laser diodes, vertical-cavity surface-emitting lasers (VCSELs) and electromagnetic radiation transmitters. In some embodiments, there may be a mixture of different LEDs or light sources within each grouping. The LEDs may also be modified to have a single or a plurality of apertures from which light emanates. This section of the device where stimulation occurs may be in close proximity to the control and power circuits. In some embodiments, the stimulation may occur may remotely. In some cases, the location and arrangement of the stimulating emitters may be connected via extension cable. In some embodiments, tissue site emitters may be connected to one another. In other embodiments, tissue site emitters may be separate from one another and connected to the control and power systems.

In some embodiments, a temperature sensor 107, an ambient light sensor 108, a microphone 110, and an accelerometer/gyroscope are included. The temperature sensor 107 may be utilized to sense ambient temperature and/or tissue temperature. The ambient light sensor 108 may be used to detect light. The microphone 110 may be utilized to sense ambient noise and/or signals. A meter 111 may be used to detect movement and/or orientation of the device. In some embodiments, the meter 111 may be an accelerometer and/or a gyroscope. The above described sensors may be utilized to determine environmental conditions that may affect the stimulation parameters delivered to the user. These inputs may be fed to the controller and the stimulation protocols may be modulated to compensate. These devices may also be used to receive user feedback and control some functions of the device. In some embodiments, microphone 110 may be used to receive voice commands. The ambient light sensor 108 may be able to sense a change in the lighting conditions that may result in a change in the visual indication displayed to the user. Tipping or shaking the device may stimulate the meter 111 to send feedback or commands to the system.

In the illustrated embodiment, visible indicators 109 are shown for each tissue site associated with the device of FIG. 1A. As with stimulating emitters, there may be any type, number, and configuration of such indicators used for the purpose of communicating information and/or augmenting the tactile sensation delivered to the user's tissue. Such indicators may be of the same or a different wavelength as those used for stimulation. As the output of these indicators is intended to be seen by the user, they will emit at least some visible wavelength. The pattern of indicator illumination may be synchronous or asynchronous with the stimulation signal in order to produce the desired effect. Such effects could include, but are not limited to, bolstering the strength of a weakly perceived tactile sensation, modifying the quality of a tactile sensation, or muting a strongly perceived tactile sensation. Proximity sensors of any sort may be placed near or within the cluster of emitters to detect the distance of the tissue to potentially modulate stimulation and/or provide feedback to a connected visualization system.

Additionally, in some embodiments, the device of FIG. 1A may employ at least one sensor including visible light cameras, infrared cameras, depth cameras, and other imaging devices to measure the conditions of the finger and/or other tissue site being stimulated. Conditions measured may include proximity to the sensors, ambient conditions surrounding the tissue, physical conditions such as callouses, scars, cuts, abrasions, bruises, finger ridges, tissue temperature, or alignment relative to the stimulating apparatus. Image analysis may be performed in such a way as to recognize these and other conditions, with the results being sent to the control program and stored. Such conditions may be used to identify the user. These conditions may also affect the tactile sensation resulting from stimulation and thus a modulation of stimulation parameters may be useful. Instructions may also be given to the user in order to improve the stimulation results.

In some embodiments, the device of FIG. 1A includes a vibration generator 113. The vibration generator 113 may include eccentric rotary mass actuators, linear resonant actuators, and piezoelectric actuators. The vibrations created by the vibration generator 113 may be utilized to alter or augment the light-induced tactile sensations. These vibrations may be transmitted through any portion of the tissue in contact with the device itself, or in contact with an object through which the device transmits the vibrations; for example, a table top on which the device sits as does the user's elbow. Vibrations may be delivered synchronously or asynchronously with light-based stimulation in order to modulate the resulting sensations in ways deemed desirable.

In some embodiments, the device of FIG. 1A may include a speaker 114 as part of the stimulation system to provide auditory augmentation and support to the delivered stimulation.

FIG. 1B illustrates alternative arrangements of the LEDs that may be utilized within the device to achieve different capabilities in stimulating the tissue consistent with embodiments of the present disclosure. In the illustrated embodiment, arrangements of one to five LED groupings 115-119 are shown, the LED groupings 115-119 are to target a single tissue site. For instance, LED grouping 115 is a single LED. LED grouping 116 is a grouping of two LEDs. LED grouping 117 is a grouping of three LEDs. LED grouping 118 is a grouping of four LEDs. LED grouping 119 is a grouping of five LEDs. In some embodiments, more individual light sources may be utilized to target a single tissue. In some embodiments, different arrangements and/or spacing between LEDs may exist. In some embodiments, different combinations of light sources within each LED grouping 115-119 may also be used. In some embodiments, LED groupings 115-119 may not include LEDs; rather they may include other electromagnetic emission devices such as, laser diodes, transmitters, or vertical cavity surface emitting laser arrays (VCSELs). Such devices may emit wavelengths of EM radiation in the visible and IR spectrum to elicit sensation.

The device may comprise a single board or multiple boards connected either physically or wirelessly. Such a system may be completely self-contained, stimulating the user's tissue without input from any other system. The system may be programmed to cycle through certain sensations without input. In some embodiments, the system may allow for a user to press at least one button on the device itself to begin a certain stimulation protocol. In some embodiments, the system may be tethered, either physically through wires, or wirelessly to another system that allows communication and control of the stimulation being delivered. In such a system, the tethered device might be a mobile phone, tablet, computer, television, game console, game controller, VR, AR, or MR device, holographic imager, or other device that is capable of communicating to the stimulation device and coordinating the stimulatory output with some other program. This may be stimulating a sensation to correlate with an image on the display of the tethered device, a sound produced, or some other abstract correlation or sensory input the tethered device presents to the user. The tethered device would send a request for a certain type of sensation to be elicited and the system would emit stimulation to elicit such a sensation. The system may also communicate how it is functioning and send other diagnostic data to the tethered device.

Groupings 115-119 of stimulation sources targeting a single tissue site may be stimulated in any order or simultaneously as the various sensations require. Stimulation parameters may be modulated in any way necessary to elicit the desired sensation. Parameters may include pulse width, frequency, duty cycle, intensity, time on at tissue, time between tissues, time between repeated stimulations, number of light sources, rate of parameter modulation, and other relevant parameters.

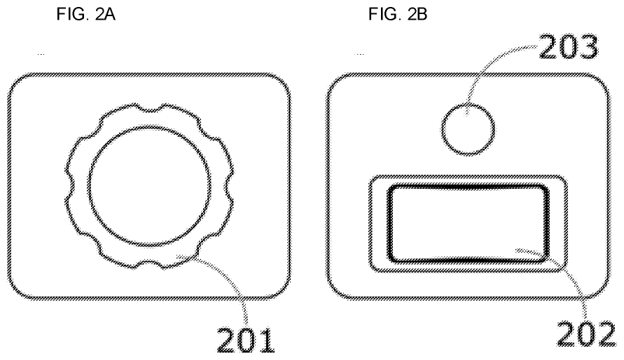

FIG. 2A illustrates a conceptual representation of a rotary encoder that may be connected to a stimulation control system and used for titration of the sensation to a user's preference consistent with embodiments of the present disclosure. Turning the rotary dial 201 clockwise or counterclockwise may indicate to the control system to increase or decrease the intensity of the sensation, to change a specific stimulation parameter, to change the quality of the sensation, or modify the output of the device in other ways. This type of control could be used in any number of ways in different programs. The rotary encoder also has a pushdown momentary switch that can convey additional commands to the stimulation controller. The push button may be used to switch stimulation modes, instruct the controller to change to a new sensation, give feedback about the user's experience, etc. Information collected from the rotary encoder can be delivered through a wired or wireless connection.

FIG. 2B illustrates an alternative conceptual representation of an end-user feedback device in the form of a rocker switch 202 and a button 203 consistent with embodiments of the present disclosure. This device configuration is capable of delivering all the same information to the stimulation controller as the rotary encoder device. Various embodiments of end-user feedback devices may be wired or wirelessly connected to the stimulation system. In some embodiments, the end-user feedback devices may include more buttons, switches, sliders, or different controls than are shown. In some embodiments, the end-user feedback devices may include less buttons, switches, sliders, or different controls than are shown. In some embodiments, end-user feedback and control may be connected to a computer, mobile phone, tablet, or other device; in these embodiments the user feedback may be sent by such devices via a variety of interfaces. Additionally, user feedback may be given to the stimulation control system by communication methods that may include voice control, eye tracking, gesture recognition, or any other methods of communication.

In some embodiments, feedback may be tracked, stored, and analyzed for the individual as well as the population of users. Feedback may include changes made by the user to customize the sensation felt, changes made by the user to the device, and/or any other type of feedback. The data collected from the feedback may be sent to a machine-learning algorithm to improve the sensation library as well as allow the system to decrease the time it takes to elicit sensation for users. User preferences as to the types of sensations found to be pleasant or otherwise desirable may be useful in modifying stimulation parameters for delivering a satisfying experience as well as informing future product offerings. Machine learning may also prove a guide to creating novel sensations as yet not discovered. In some embodiments, associations of visual and auditory features that most closely correspond to certain tactile sensations may also be tracked and analyzed using machine learning algorithms and other forms of data analysis. Based on the results of the analysis, an automated generation of tactile stimulation parameters and software and hardware configurations may result to deliver an optimized end user experience.

The finger stimulator sleeve may provide certain advantages. For example, the user may place her hand anywhere in space and the finger stimulator sleeve remains in the same position relative to the finger surface. This may offer a range-of-motion freedom with few restrictions on the working area in which the stimulation system may work.

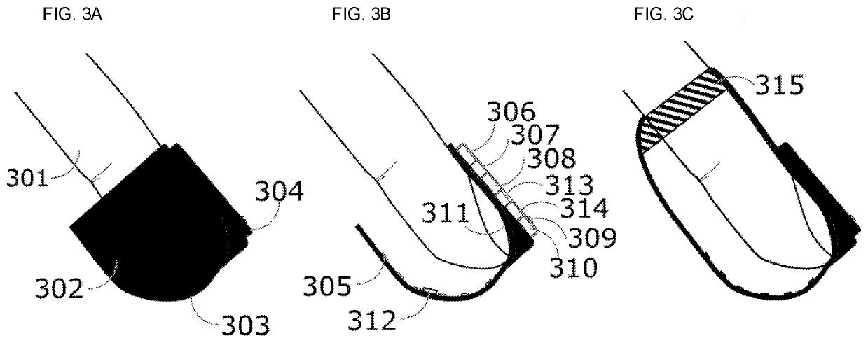

FIG. 3A illustrates a conceptual representation of a finger sleeve device system with a finger sleeve device attached to a user's fingernail consistent with embodiments of the present disclosure. This device may work singularly or together with other devices, including other finger stimulator sleeves, VR, AR, and MR headsets, wearable devices, gaming consoles, gaming controllers, mobile phones and devices, computers, and holographic imaging systems. The user's finger 301 is inserted into the sleeve 302 allowing at least a portion of the distal phalanx to be surrounded by the device. The tip of the sleeve 303 may optionally be coated such that it is capable of interacting with various other systems. For example, capacitive coating may be applied to sleeve 303 for activating touch screen devices and fiducial or reflective markers for determining positioning in 3D tracking system. In some embodiments, there may be an active device such as a transmitter, light source, or ultrasound emitter used in conjunction with one or more appropriate receivers to show the location of the sleeve in space. A power supply and control system are located in a housing 304. In some embodiments, the power supply and control system may be located separately from the finger sleeve and power and control information may be delivered via physical wires and/or wirelessly.

FIG. 3B illustrates a cutaway view of the finger sleeve device of the above described FIG. 3A and illustrates that the sleeve that is in contact with the dorsal side of the user's finger and separated from the palmar side of the finger consistent with embodiments of the present disclosure. The illustrated embodiment comprises a sleeve in contact with the dorsal side of the user's finger while maintaining no contact with the palmar side of the finger. In some embodiments, the contact or anchor point of the finger sleeve on the dorsal side of the hand may be the finger nail(s). In some embodiments, the contract or anchor point of the finger sleeve may be in a different location.

Facing the palmar side of the finger is an array or a matrix of stimulating light sources 305. There may be any number of such emitters employed. The stimulating light sources 305 may include any type of light emitter capable of eliciting sensation, including light emitters described elsewhere in this disclosure. In some embodiments, a mixture of different light transmitters may be utilized in each finger stimulator sleeve. The light emitter source may also be modified to allow for an aperture to limit the incident light to a desirable spot size on the user's tissue. The modification of the light emitter may be a coating or sheet that is opaque to minimize light leakage exposure onto the user's target tissue. In some embodiments, the modification of the light emitter may also comprise a highly reflective material that reflects light. In some embodiments, there may be one aperture. In other embodiments, there may be a plurality of apertures to allow the electromagnetic radiation to escape and be directed onto the user's tissue. The apertures may be a single type of emitter and/or several types of emitters. The apertures may be arranged in a variety of arrangements.

In the illustrated embodiment, a control system and other supporting devices are contained in the housing on the dorsal side of the finger directly above the nail. The device may include a power supply 306, a microprocessor 307, a light source driver 308, and other electronic components 310. The power supply 306 may include a battery. The other electronic components 310 may include voltage regulator, current regulator, memory chips, communication chips, and graphic processing units. The microprocessor 307 may control the stimulation system with no external inputs. In some embodiments, the microprocessor 307 may coordinate the stimuli with an externally connected system, such as, a computer, video projection system, game console, VR, AR, and MR device, mobile phone, holographic imager, and game controller. In some embodiments, the microprocessor 307, power supply 306, and other electronics 310, including memory chips, graphic processing units, wireless communications chips, or application specific integrated circuits (ASICs), may reside remotely from the stimulating LEDs 305. In these embodiments, power and communication may be accomplished via wire and/or wirelessly. In some embodiments, an indicator light 309 may be utilized to visually display information to the user. This may be a single color or multicolor emitter such as an LED that lights up in various ways to provide the user information and/or to augment the tactile sensory stimulation. In some embodiments, a vibration generator 313 such as an eccentrically rotating motor or linear actuator may be mounted to the finger sleeve. The vibration generator 313 may be controlled by the control program to synchronously or asynchronously work with the light sources to modify the tactile sensation that is stimulated by the stimulating LEDs 305. In some embodiments, a speaker 314 may be part of the light-based stimulation system to provide auditory augmentation and support to the delivered stimulation.

In some embodiments, the device may employ at least one sensor 312, such as visible light cameras, infrared cameras, and other imaging devices, to measure the conditions of the finger and/or other tissue site being stimulated. Conditions measured may include proximity to the sensors, physical conditions such as callouses, scars, cuts, abrasions, bruises, temperature, or alignment relative to the stimulating apparatus. Image analysis may be performed to recognize these and other conditions and the results fed to the control program. Such conditions may affect the tactile sensation resulting from stimulation and a modulation of stimulation parameters may be useful. Instructions may also be sent to the user in order to improve the stimulation results.

The finger sleeve may be attached to the user's fingernail at 311 with an adhesive that allows for a stable positioning while also being removable and reusable. In some embodiments, the adhesive may be a replaceable film. In some embodiments, a permanent coating may be utilized. The attachment to the nail allows for a space to be maintained between the stimulating surface of the sleeve and the user's finger on the palmar side. There may be other embodiments where the sleeve makes contact over the stimulating surface as well.

FIG. 3C illustrates an example attachment scheme that may or may not include adhesive over the nail. In the illustrated embodiment, an additional point of attachment where the intermediate phalanx is encircled by the open end of the sleeve 315 such that the sleeve holds in place on the finger. The finger sleeve may be used to directly stimulate the finger with no interactive commands, or to stimulate without coordination of any outside system. In some embodiments, the finger sleeve may be coordinated with some other system to convey information such as, but not limited to, touch, texture, or other tactile quality. For example, if the sleeve was capable of interacting with a capacitive touch screen, then the image displayed on a screen may have some tactile properties associated with touching that object. In some embodiments, the sleeve making contacting with display may cause the device driving the display to communicate to the stimulation controller to deliver a certain type of stimulation to the finger. Once the sleeve is no longer in contact with the display, the lack of contact is communicated to the stimulation controller and the stimulation stops. In another embodiment, a non-contact interface may allow without physical contact between the sleeve and the display, a gesture recognition system and motion tracking system to determine the timing and type of tactile stimulation requested. The timing of the various sensations may require different beginning and ending times relative to contact with the display. Therefore, they are not always strictly temporally linked, but the coordination exists to make the sensation correlate to the interaction of what is shown on the display.

In some embodiments, a finger without a stimulation sleeve may interact with a touch sensitive display and/or through the non-contact interface. The interaction may be delivered to other finger(s) wearing the finger stimulation sleeves. In this case the stimulation is not directed onto the same finger that is interacting with the object on the display. Instead, the sensation may still be induced in a similar way but onto the other finger(s) that have the stimulation sleeves. Here, the stimulated finger is a surrogate for the finger interacting with a displayed object.

The finger sleeve shown may be utilized in any number of augmented, virtual, or mixed reality systems, or with a holographic imaging system. When interacting with a virtual object, the system may define the type of tactile feedback delivered. As the finger comes into contact or close contact with the surface of a virtual object, the system may send a signal that contact has been made and requests a certain type of sensation or stimulation protocol to be delivered. The systems may request sensation and intensity based on the virtual object. Such systems may further decide how virtual objects react to particular touches, grabbing actions or other interactions. These systems may also request stimulation to be delivered in the absence of interaction with a virtual object to achieve any number of tactile effects.

In some embodiments, all ten fingers may utilize finger sleeves simultaneously. In some embodiments, stimulation may be delivered to other body parts, including a palm, wrist, forearm, leg, back, and/or any other body part. In some embodiments, stimulation may be coordinated wirelessly between the sleeve devices. In some embodiments, a central unit worn on or near the hand or elsewhere on the body may plug in communication and/or power wires between the devices. When paired with some other external system, the coordination of stimulation may depend on the connection to that system or coordinated within the network of sleeve devices.

A glove for tactile stimulation offers a number of advantages. The user may place her hand anywhere in space and the glove remains in the same position relative to the hand. The glove offers a feeling of freedom in terms of range of motion with few restrictions on the working area of a stimulation system. There is no need to aim an optical stimulation relative to the target tissue. The tissue may be held constantly at the same distance relative to the optical output or aperture and may eliminate the need for an adaptive focusing element. Here are presented several types of gloved applications.

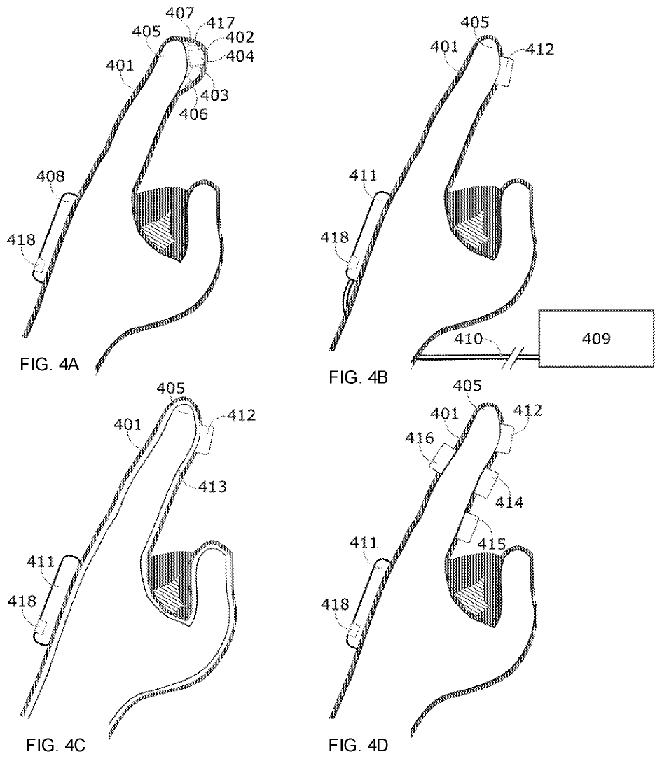

FIG. 4A illustrates an example conceptual representation of a glove comprising a light-based tactile stimulation system consistent with embodiments of the present disclosure. A cutaway side view of a glove 401 is shown with an enlarged pocket 402 for a stimulation system at the palmar side of the distal phalanx 405 that contains the optical source 403 and the steering system 404. The optical source 403 may emit one or more wavelengths independently or simultaneously. The optical source 403 may comprise at least one light source such as, light emitting diodes (LEDs), infrared emitters and LEDs, laser diodes, and vertical cavity surface emitting lasers (VCSELs). The optical source 403 may also be modified to allow at least one aperture that light may emanate from. In some embodiments, the optical source 403 may be located more proximal to the user and the light may be delivered to the distal phalanx 405 through a fiber optic cable. The beam steering system 404 may comprise any type of device such as optical, mechanical, electrical, or MEMS. For example, mirrored MEMS devices, DMD used in DLP projectors, and spatial light modulators (SLMs). A tracking module 406 represents accelerometer and/or gyroscopic devices utilized in aid of tracking the finger position in space. There may be one or more such devices to track at least one desired tissue location. In some embodiments, a vibratory module 407 may induce vibrations of various amplitudes and frequencies that augment the optical tactile stimulation. In some embodiments, a speaker 418 may be part of the light-based stimulation system to provide auditory augmentation and support to the delivered stimulation.

The control module 408 may control the glove 401 autonomously or in connection with other systems. The system may communicate wirelessly with other devices connected to the optical control system. This control module 408 may coordinate signals received from the connected system, sending stimulation commands and power to the optical source 403, the beam steering module 404, the vibratory module 407, and receive information from the tacking module 406 and any other sensors such as, cameras, temperature sensors, and photo detectors. The control module 408 may include a battery or other mobile power supply. The connections between all these parts may be wired or wireless. The user's palmar side skin surface of the distal phalanx does not come into contact with any glove components in the illustrated embodiment due to the structural support integrated into the fingertip portion of the glove. In some embodiments, a surface may be placed in direct contact with the skin. In another embodiment, the devices may employ a multitude of light sources placed in close proximity to the skin eliminating the need for a beam steering system 404 or optical fibers to direct the stimulating light.

In some embodiments, the device may utilize at least one sensor 417, such as visible light cameras, infrared cameras, and other imaging devices to measure the conditions of the finger and/or other tissue site being stimulated. Conditions measured may include proximity to the sensors, physical conditions such as callouses, scars, cuts, abrasions, bruises, temperature, or alignment relative to the stimulating apparatus. Image analysis may be performed to recognize these and other conditions and the results fed to the control program. Such conditions may affect the tactile sensation resulting from stimulation and a modulation of stimulation parameters may be useful. Instructions may also be given to the user in order to improve the stimulation results.

FIG. 4B illustrates an example conceptual representation of a glove 401 comprising a light-based tactile stimulation system wherein a contact interface for stimulation of the index finger consistent with embodiments of the present disclosure. In the illustrated embodiment, a main optical control system 409 may include the control system, power supply, optical source, communication modules and other electronic components, such as, communication chips, memory chips, GPUs, and ASICs. This is connected to the glove 401 through a cable 410 that may contain electrical wires and/or optical fiber that run to the glove 401. Cable 410 connects to a module 411 on the glove 401, or another location, that serves to distribute the connections as necessary to the various components, such as, but not limited to, those located on the distal phalanx. In some embodiments, the module 411 may contain a circuit board and/or processor to facilitate communication to and from sensors and actuators. The module 412 is the optical stimulation interface the light passes and stimulates the user's finger 405. Module 411 and stimulation module 412 are also connected by optical fiber not seen in this orientation. Stimulation module 412 may direct a single beam or multiple beams of light onto the tissue. It may be a passive or active device. All modules and sensors represented in the wireless embodiment shown in FIG. 4A may also be implemented in a wired embodiment such as the embodiment shown in FIG. 4B.

FIG. 4C illustrates an example conceptual representation of a glove 401 comprising a light-based tactile stimulation system wherein a stimulation module 412 is separated from the skin by inflating, inserting internal supports consistent with embodiments of the present disclosure. Minimizing contact with the user's tissue accomplishes the goal of eliminating or minimizing other tactile sensations that are not intentionally introduced by the stimulation system.

FIG. 4D illustrates an example conceptual representation of a glove 401 comprising a light-based tactile stimulation system in which a plurality of stimulation modules 412-416 are placed in several locations on a single finger consistent with embodiments of the present disclosure. Stimulation systems may be placed on any portion of the user's skin in contact and/or non-contact configurations. These stimulation systems are coordinated with a controller either with a wired and/or wireless interface. Such a system might be used with VR, AR, and MR devices, and holographic imaging systems. In some embodiments, optical stimulation may be utilized with another tactile stimulation system in the glove 401, such as a rotating eccentrically loaded motor commonly used for vibration. In some embodiments, vibration may be provided by the mechanical movements created as a byproduct of some types of beam steering systems. In such embodiments, the beam steering system may be vibrationally isolated on a platform which may be optionally mechanically coupled and uncoupled from portions of the glove 401 that vibrations may be transmitted at various intensities as controlled by the stimulation system. Time-varying disturbances including velocity and acceleration of the beam steering system may be delivered in a periodic and steady-state fashion, a transient input, or a random input. The periodic input may be a harmonic or a non-harmonic disturbance.

The glove 401 may require power that may be supplied by either a battery or a power supply run to the glove 401 through a cable. Communication between a control module and the glove 401 may be accomplished wirelessly or by cable. The control system may communicate with any device, including, but not limited to, a computer, tablet, phone, gaming console or controller, VR, AR, and VR headset or devices, dedicated systems, or any other capable device.

Hand position may be an important variable for optical tactile stimulation. The position of the hand in space may be monitored by external cameras or sensors embedded in the glove 401. For an embodiment with external cameras or sensors, hand tracking may be accomplished with or without fiducial markers on the surface of the glove 401. Accelerometers and gyroscopic devices may be employed for any portion of the glove 401 or in multiple positions. In some embodiments, temperature sensors measuring the ambient temperature and/or the user's tissue temperature may be incorporated into the glove 401 to allow the control system to modulate the stimulation accordingly.

In some embodiments, the glove 401 exists where the portions of the hand are exposed and the tactile stimulation system is external to the glove 401. The portions of the glove 401 that cover the hand can be used to more easily or reliably tracking the tissue position in space. The controller then directs the stimulation onto the tissue not covered by the glove 401.

In some embodiments, the glove device utilizes visible indicators near the fingertips and anywhere else on the glove 401 to send feedback to the user. This feedback may be to augment the sensation or give additional information to the user about the functioning of the device. These indicators may be single or multiple wavelengths, wherein at least a portion of the wavelength is visible to the eye.

A computer mouse is a common tool for interaction with computers and other devices and machines and, thus, the computer mouse may be a useful device for a tactile stimulation system. It is advantageous because the fingers are frequently used to manipulate the mouse and are readily available for stimulation. Additionally, the mouse cursor on the computer's display may act as a surrogate for the user's finger when moving over an object on a display about which some tactile information may be conveyed to the user. A previous disclosure has described an embodiment of a tactile system stimulating a user's finger through a transparent window on the mouse key. In some embodiments, the user does not directly contact a surface where stimulation occurs.

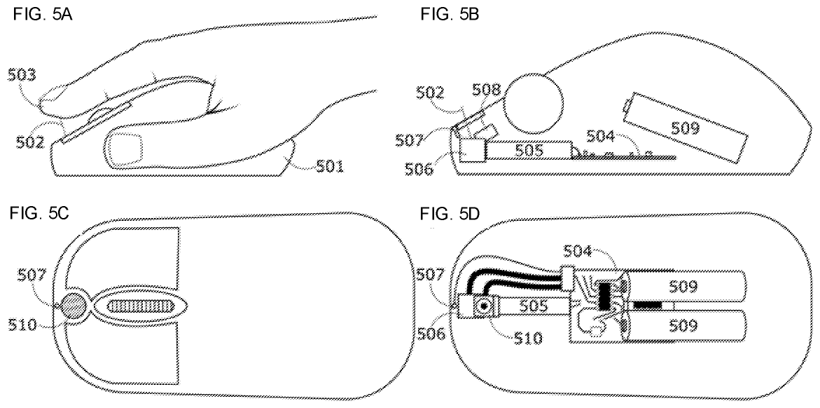

FIG. 5A illustrates a conceptual representation of a computer mouse 501 that includes a tactile stimulation system consistent with embodiments of the present disclosure. The system generates and steers a beam of optical energy 502 onto a targeted finger 503. This system communicates with the computer or another system to allow some coordination with what is displayed on the screen.

FIG. 5B illustrates a side-view cutaway view of a computer mouse and some of the internal components associated with a tactile system consistent with embodiments of the present disclosure. A circuit board may comprise components to communicate with the computer, control the laser source 505, and the beam steering system 506. The laser source 505 may comprise a single wavelength or multiple wavelengths. The laser source 505 may be focused in any variety of ways. The beam produced enters the beam steering system 506 where it is directed onto the tissue in the desired fashion. The beam steering system 506 may comprise mechanical or solid-state components that may reflect or bend the beam to steer it. Such components may include mirrors, prisms, digital micromirror devices (DMD) used in digital light processing (DLP) projectors, fiber optic cables, or spatial light modulators. Actuators, such as, motors, MEMS devices, and DMD used in DLP projectors, may be utilized to mechanically or electromechanically move components to properly steer the beam. In some embodiments, there may be multiple light sources such as, LEDs arranged in a manner such that each individual light source may be turned on and off rather than steering a single beam. To ensure the finger is in place before emitting the optical stimulation, an optional proximity sensor 507 may be positioned near the aperture 510 through which stimulation is directed. In some embodiments, the system may be powered by battery 509. In some embodiments, the system may be powered by a USB cable connected to the computer, or another power source such as a wall outlet.

FIG. 5C illustrates a top view of a computer mouse wherein an aperture 510 for optical stimulation and a proximity sensor 507 are visible, consistent with embodiments of the present disclosure. The aperture 510 may be open to the air or may be a window that is transparent to the wavelength of light used for stimulation. In some embodiments, the aperture 510 may be a focusing lens to control the spot size of the light onto the user's finger.

FIG. 5D illustrates a top-view cutaway of the internal components of a computer mouse consistent with embodiments of the present disclosure. In some embodiments, the device may utilize at least one sensor such as, visible light cameras, infrared cameras, and other imaging devices to measure the conditions of the finger and/or other tissue site being stimulated. Conditions measured may include proximity to the sensors, ambient conditions surrounding the tissue, physical condition such as callouses, scars, cuts, abrasions, bruises, temperature, or alignment relative to the stimulating apparatus. Image analysis may be performed to recognize these and other conditions and the results fed to the control program. Such conditions may affect the tactile sensation resulting from stimulation and a modulation of stimulation parameters may be useful. Instructions may also be sent to the user in order to improve the stimulation results.

When used with a visualization system, such as, VR, AR, and MR headset or devices, holographic imaging systems, computer monitor, interactive display or other vision display systems, the tactile sensations may be spatially and temporally aligned with the visualization. In a VR embodiment, any body part the user is expecting to have an interaction with may be included visually. In many cases the hand may be shown in the virtual space along with any objects available for interaction. When the hand virtually contacts a virtual object the proper spatial and temporal coordination with the tactile stimulation system may enhance the sense of reality by providing tactile feedback. Some stimulation protocols and methods have an inherent lag between the start of stimulation and the onset of a perceived sensation. In such cases a predictive algorithm may be employed to anticipate contact with the virtual object and preemptively begin stimulation. If the predicted contact does not occur, then the stimulation may be ceased in sufficient time to avoid or minimize any tactile sensation being felt by the user. In some embodiments, the virtual object may be manipulated such that the object does not react to the apparent contact until the time required for tactile sensation onset has elapsed. For example, in the case of sliding a box across a tabletop the box would not begin to move until the time that the tactile sensation was expected. Communication between the stimulation and the visual system may accomplish these goals.

Interaction with objects in virtual space may further require coordination of the space where both the virtual system and the tactile stimulation system may operate. Various limitation on the physical space in which the user may interact must be addressed so that the two systems may work together. The stimulation system may have a working area that requires that the VR, AR, or MR system only place objects available for interaction within that working area. This ensures that the user does not reach out beyond the physical space and expect a tactile stimulation that the system may be unable to provide.

The VR, AR, or MR system and holographic imaging systems must also communicate with the tactile stimulation system, the area and characteristics of the virtual objects to the stimulation system so that the appropriate tactile stimulation protocols are used within a constrained physical space correlated with the objects. As an example, a small cube in space may comprise only a small portion of the working area of the tactile stimulation system, but if a user places her hand within the eworking area of the tactile stimulation system but away from the cube she may not expect to have any tactile sensation. As the user moves her hand to virtually touch the cube, she may expect stimulation and may have an expectation of what the cube might feel like based on its visual appearance. The virtual hand and the virtual cube may be in contact, or share the same virtual space. The stimulation system actively stimulates the appropriate portion of the user's hand to induce the expected sensation.

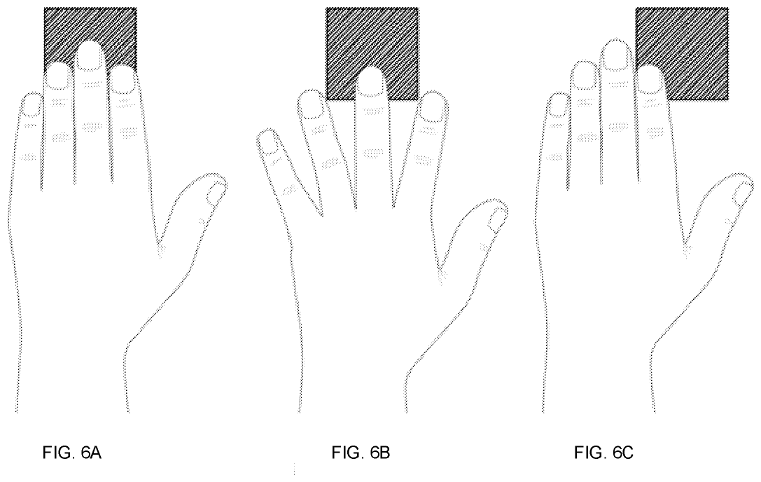

FIG. 6A illustrates a conceptual representation of a virtual hand in contact with a virtual cube, in which fingers L2 (left index), L3 (left middle) and L4 (left ring) are in contact with a virtual cube. Each finger may receive sufficient stimulation to elicit sensation felt across all 3 of these fingers consistent with embodiments of the present disclosure. This may be accomplished with a single light source being directed alternately between each of the fingers, or with multiple light sources each directed at one or more fingers.

FIG. 6B illustrates a virtual hand a virtual cube, the L3 remains in virtual contact with the cube and would therefore be the only finger to receive stimulation consistent with embodiments of the present disclosure. Fingers L2 and L4 are spread apart and the hand moved down relative to the virtual cube sufficiently that they are no longer in the same space as the virtual cube and therefore should not be stimulated.

FIG. 6C illustrates a virtual hand in contact with the virtual cube, wherein the fingers are in close proximity and the hand is moved to be laterally relative to the cube that L2 remains in virtual contact with the cube and is the finger to be stimulated consistent with embodiments of the present disclosure.

In some embodiments, the laws of physics may not apply to objects in a virtual world. Similarly, tactile sensations may not mimic reality or respond as a user may intuitively expect. The tactile sensations and physics model interactions associated with an object may be selected by the tactile designer and/or tactile content generator of an experience. The tactile designer and/or tactile content generator may select behaviors that are expected or that are unexpected. For example, a user may bring his hand toward a cube from the side moving laterally expecting to feel some type of pressing sensation on his finger and for the cube to move in the direction of his hand's movement. However, the designer may choose to have the cube move vertically or disappear while inducing a gentle warming sensation on the palm.

In some embodiments, coordination of an audio input to the user and tactile stimulation may exists in systems such as, VR, AR, and MR systems, holographic imagers, computers, and mobile devices. Audio input may be delivered to the user utilizing a variety of methods, including headphones and speakers. Auditory signals may enhance or diminish the tactile sensations. The auditory inputs may be synchronous or asynchronous to the tactile stimulation being delivered to the user in order to achieve certain sensory experiences. In one example, a sound consistent with gliding of the user's hand across a virtual object or a real physical object may be heard and amplified to the user. Such sounds may mimic real world sounds. In some embodiments, the sounds may be different from real world sounds. The auditory components may be chosen to enhance, distract, or detract the user from the sensory experience.

In some embodiments, the system may be utilized with VR systems, AR systems, MR systems, holography, and other 3D or volumetric displays. One of these technologies may utilize a light-based tactile stimulator to allow for cutaneous sensations in concert with visualizations provided. For example, a user may see a virtual box placed in her visual field. A hand tracking system follows the movement of her hand in front of her face, placing a virtual representation of the hand in the visualization. As the virtual finger comes into contact with the virtual box, the tactile stimulation system directs one or more beams of light onto the user's finger and induces a sensation. Such a sensation may mimic the sensation of a real box, or may be programmed to feel different than a real box.

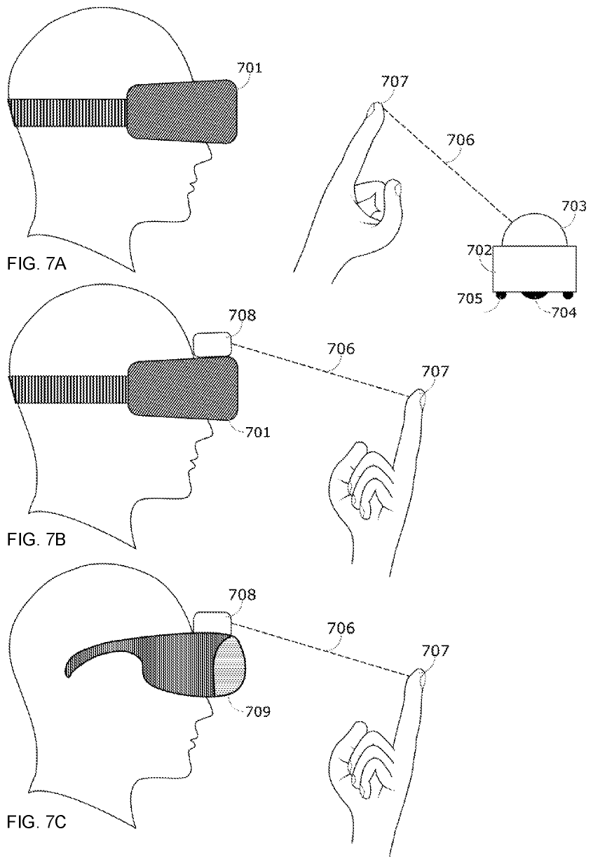

FIG. 7A illustrates an example of a user wearing a VR headset 701, that is associated with a tactile stimulation system 702 consistent with embodiments of the present disclosure. The headset 701 along with hand tracking allows the user to see a virtual scene as well as a virtual representation of his hand. As the user's hand virtually contacts an object the stimulation system 702 directs a beam of light 706 such as, a collimated laser, or multiple beams of light of the same or different wavelengths, onto the appropriate area of the hand, the area of the hand in contact, and elicits the desired sensation. Housed in the dome 703 is a mechanism to direct the one or more beams of light 706 onto the target tissue in such a way that sensation is elicited. The stimulation system 702 may be placed anywhere relative to the user to elicit sensations in space relative to the user and to the virtual world. This includes, but is not limited to, a drone carrying the tactile stimulation system 702, a mobile and portable wheeled platform, or a stationary or fixed stimulation system. Shown herein is a mobile system where wheel 704 is one of two driven wheels that can move the stimulation system 702 in any direction on a smooth horizontal surface and wheel 705 is a caster wheel to stabilize the device. In some embodiments, the mobile system may include multiple wheels similar to wheel 705. The mobile system may be placed on any surface where it may move to properly stimulate a user's tissue. The mobile system may move on a wheeled chassis may move, for example, across floors, tabletops, counters, and so forth. The mobile system may include a battery and/or a power cord. The movement of the mobile system may be coordinated with the optical stimulation control unit and with the VR system.

FIG. 7B illustrates an example tactile stimulation system 708 attached directly to the VR headset 701 and worn by the user allowing stimulation directed outward from the user's body onto his peripheral tissue. In this figure, the tactile stimulation system 708 is attached directly to the VR headset 701. The stimulation system 708 directs a beam of optical energy 706 onto the user's finger 707. In the illustrated embodiment, the virtual reality system and the tactile stimulation system are separate devices. In some embodiments, a single unit comprises the virtual reality system and the stimulation system. Such an embodiment would direct the light from the single unit.

FIG. 7C illustrates a tactile stimulation system that may be mounted to goggles 709 including AR goggles, VR goggles, MR goggle, or other non-vision occluding projection systems consistent with embodiments of the present disclosure. In such embodiments, the apparatus 709 may allow for real objects and/or virtual objects to be seen by the user.

In the illustrated embodiment, the virtual reality system and the tactile stimulation system are separate. In some embodiments, a single unit comprises the virtual reality system and the tactile stimulation system. Such an embodiment may direct the light from the single unit.

In some embodiments, stimulation may be provided to any portion of the user's skin, the stimulating beam may be directed to any portion of the skin to allow for interaction with the virtual reality system. For example, a warm breeze blowing past the user's cheek, an insect landing on a user's forearm, and a dog's tail brushing a user's leg.

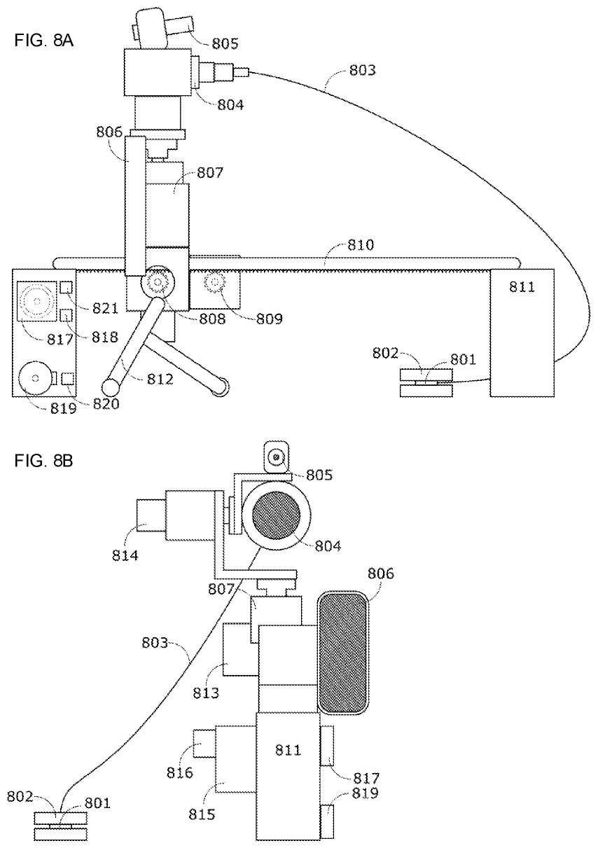

FIG. 8A illustrates a side-view diagram of a non-contact, free-space tactile stimulation system consistent with embodiments of the present disclosure. The system comprises two light sources 801, 805 and the support structure, sensors, and actuators to direct the light onto a user's tissue, including, but not limited to, the hand and arm. A light source 801 may be between two metal heat sink plates 802. The light source 801 is powered by a power supply. The beam from the light source 801 is directed through a fiber optic cable 803 to an optic assembly 804 that expands, collimates, and focuses the beam. The second light source 805 is mounted above the first optic assembly to allow the beam from the second light source 805 to coincident with the beam of the first light source 801 at a point in space. The second light source 805 may be focused down at a prescribed focal length or may be a highly collimated laser beam. The point of coincidence of the beam for the first light source 801 and the beam for the second light source 805, may be the point utilized to stimulate the user's hand. In some embodiments, a different point(s) may be utilized to stimulate the user's hand. In some embodiments, the wavelengths may be the same for the beams associated with the first light source 801 and the second light source 805. In some embodiments, the wavelengths may be different for the beams associated with the first light source 801 and the second light source 805.

The distance where the beam coincides can be determined based on the focal length of the focusing lens that is part of the optic assembly 804. However, other positions within the beam's path of one or both of the light sources can be used for stimulation. In some embodiments, multiple beams may be combined and emitted from the same optics 804. In some embodiments, the beams may be multiple wavelengths. In the illustrated embodiment, light source 805 is a tightly collimated beam. When a user places her hand in the workspace, which may be left of the light emitting elements 804 and 805, a hand tracking device 806 creates a mathematical model of the hand in space and feeds the tissue locations into the software used to direct the beams. The system may be capable of locating the target tissue site in space relative to the light source. Housing 807 surrounds the pan servo, which pans the light sources horizontally. There is a tilt servo behind the optics 804 that moves the optics vertically. The system moves on a beam 810 with a rack gear using a multi-turn servo attached to a pinion gear 808. This moves the system either closer to or farther from the tissue as necessary to align and or focus the light from either or both of the light sources. Position along this beam, called the z-axis, is determined by an analog reading from a multi-turn potentiometer attached to a pinion gear 809 that turns as the carriage moves. The beam may be mounted on either side by pillars 811 to an optical breadboard for rigidity. Smaller beams attached to bearings 812 provide support that moves along with the carriage to minimize shaking of the carriage assembly to improve positional accuracy.

FIG. 8B illustrates a front view of the optics of the non-contact, free-space tactile stimulation system consistent with embodiments of the present disclosure. A fiber optic cable 803 may direct the light to an optic assembly 804. The optic assembly 804 includes a final focusing lens. The second optical source 805 includes a housing and collimating lens in the center. In the illustrated embodiment, a front face of a hand-tracking device 806 is shown. The non-contact, free-space tactile stimulation system may include a pan servo 813, a tilt servo 814, a z-axis servo 815, and a potentiometer 816. In some embodiments, the two axes are centered. The attachment of the tilt assembly via and L-bracket to the pan assembly may allow the tilt rotation to be centered directly over the axis of pan rotation. In some embodiments, different arrangements may be utilized to track a tissue location, and direct one or more beams of light in three dimensions.

In some embodiments, an auditory module, such as a speaker 817 or headphone, may be part of the light-based stimulation system to provide auditory augmentation and support to the delivered stimulation. A vibration generator 819, such as an eccentrically loaded motor or linear actuator, may also be optionally present to provide augmentation and support the tactile sensation. Sensors such as, a microphone 818 and accelerometers 820 may be utilized to measure surrounding sound and vibration conditions to adjust the auditory and vibration signals delivered to the speaker 817 and vibration generator 819 to achieve the desired tactile sensation. In some embodiments, an optional light sensor 821 may be employed to measure ambient lighting conditions that may affect the system. The light sensor may include photo-emissive cells, photoconductive cells, photovoltaic cells, photo-junction devices, or cameras. Stimulation parameters may be modulated to compensate for certain lighting conditions. In some embodiments, three or more light sources may be used. In some embodiments, one light source may be used.

While specific embodiments, examples, and applications of the disclosure have been illustrated and described, it is to be understood that the disclosure is not limited to the precise configurations and components disclosed herein. Accordingly, many changes may be made to the details of the above-described embodiments without departing from the underlying principles of this disclosure. The scope of the present invention should, therefore, be determined only by the following claims.

What is claimed:

1. A sensory stimulation system, comprising:
an optical stimulation system to:
generate an output operable to excite neural tissue;
induce a tactile sensation in a user of an electronic device based upon a tactile application executable on the electronic device; and
generate a simulated object;
an interface component to selectively direct the output of the stimulation system onto a target area; and
a controller in communication with the optical stimulation system and the interface component to generate a control signal to cause the optical stimulation system to modify one or more characteristics of the output of the stimulation system to induce a tactile representation of the simulated object.

2. The sensory stimulation system of claim 1, wherein the target area of the system is an area of skin of a user.

3. The sensory stimulation system of claim 1, wherein the output operable to excite neural tissue is a beam of light.

4. The sensory stimulation system of claim 1, wherein the characteristic of the output of the stimulation system is the wavelength of the output.

5. A system, comprising:
a processor; and
a non-transitory computer-readable medium with instructions stored thereon that, when implemented by the processor, causes the system to perform operations for stimulating a sensation, the operations comprising:
receiving data associated with at least one of a target area and a simulated object;
determining the one or more target areas to direct an output operable to excite neural tissue;
generating the output operable to excite neural tissue to be directed at one or more target areas;
generating the simulated object; and
directing the output operable to excite neural tissue to the determined one or more target areas.

6. The system of claim 5, further comprising determining a time to direct the output operable to excite neural tissue at the one or more target areas.

7. The system of claim 5, wherein the output is a beam of light.

8. The system of claim 5, wherein the data received describes one or more target areas.

9. The system of claim 5, wherein the data received describes the object to be simulated.

10. The system of claim 5, wherein the output is generated to the determined location to induce a tactile sensation.

11. The system of claim 5, wherein the output is generated at the determined time to induce a tactile sensation.

12. A method, comprising:
determining, by an interface component, one or more targeted areas to direct an output operable to excite neural tissue;
generating, by an optical stimulation system, the output operable to excite neural tissue to induce a tactile sensation in a use of an electronic device based upon a tactile application executable on the electronic device;
generating a simulated object;
communicating, by a controller, between the optical stimulation system and the interface component to generate a control signal; and
modifying, by the control signal, one or more characteristics of the output the stimulation system to modify one or more characteristics of the output of the stimulation system to induce a tactile representation of the simulated object.

13. The method of claim 12, wherein the output is a beam of light.

14. The method of claim 12, wherein a characteristic of the output is a wavelength.

15. The method of claim 12, further comprising determining a time to direct the output at the target area.
